(19) United States
(12) Patent Application Publication
(10) Pub. No.: US 2003/0211617 A1
(43) Pub. Date: Nov. 13, 2003

(54) Blood glucose meter that reminds the user to test after a hypoglycemic event

(75) Inventor: Steven Paul Jones, Rochester, MN (US)

Correspondence Address: Robert R. Williams, Patent Agent, IBM Corporation, Dept. 917, 3605 Highway 52 North, Rochester, MN, 55901-7829, US

(73) Assignee: International Business Machines Corporation, Armonk, NY

(21) Appl. No.: 10/140,547

(22) Filed: May 7, 2002

(51) Int. Cl. G01N 33/66

(52) U.S. Cl. 436/14; 436/95; 436/149; 436/150; 436/164; 435/14; 422/68.1; 422/82.02; 422/82.05; 702/19; 702/31

(57) ABSTRACT

Methods, program product, and apparatus are provided for implementing a blood glucose meter that will remind the user to test his or her blood glucose after a programmable wait when a hypoglycemic event is detected. Diabetics frequently have a “rebound” hyperglycemic event (high blood glucose) occur as a result of a hypoglycemic event (low blood glucose). The disclosed invention allows the user to program the meter with a waiting period which he or she determines is appropriate to wait following a low blood glucose reading. At the end of this period, the meter will alert the user by way of an audible or tactile warning.

### FIELD OF THE INVENTION

The present invention relates to blood glucose meters, and in particular, to an inexpensive blood glucose meter that reminds the user to recheck his or her blood glucose after a programmable interval when the meter detects a hypoglycemic event.

### DESCRIPTION OF THE RELATED ART

Insulin dependent diabetes mellitus (IDDM) is caused by the autoimmune destruction of the insulin producing islets of Langerhans in the pancreas. Insulin replacement therapy is the interim treatment for IDDM until such time as islet transplants, stem cell treatments, or other improved treatments become feasible. Insulin lowers the concentration of glucose in the blood, while food—in particular, carbohydrates—raises the concentration of glucose in the blood. The challenge of insulin therapy is to administer food and insulin in a manner that maintains blood glucose concentrations in an acceptable range, thereby avoiding hypoglycemia and hyperglycemia.

Hyperglycemia (high blood glucose concentration) has adverse long-term consequences for the body. These consequences include kidney damage leading to kidney failure, microaneurisms in the retina causing blindness, and the blocking of capillaries in the extremities causing an inability to heal wounds and subsequent gangrene. Hypoglycemia (low blood glucose concentration) has an immediate adverse consequence of reduced brain function that leads to confusion and an inability to reason, remember, or react. In the extreme, hypoglycemia causes seizure, coma, and death.

The first insulin used by diabetes patients was regular insulin taken from beef or pig pancreases. This insulin lasts for about six hours, so that patients were required to inject it three or four times per day. After World War II, longer acting insulin was developed by binding regular insulin to protamine and zinc. Regular insulin dissociates slowly from protamine and zinc, extending insulin action to twelve hours for intermediate acting insulin and twenty-four hours for ultralente insulin. Patients enjoyed reducing injections to one per day, but were required to modify their eating to a snack-all-day regimen to avoid hypoglycemia. The one daily insulin dose was adjusted as needed to reduce the incidence of both hypoglycemia and hyperglycemia.

The development of portable blood glucose meters encouraged the development of more sophisticated insulin therapy regimens. One of these regimens is the split/mixed regiment that consists of two daily doses of mixed regular and intermediate acting insulins taken before breakfast and dinner. These four insulin therapy components are adjusted using blood glucose values measured before each meal and at bedtime. Patients using the split/mixed regimen are required to eat substantially the same meals every day so that the four insulin components may be adapted to the consistent meal pattern over time. Patients on the split/mixed regimen are not only faced with a consistent pattern of what they eat in terms of amount of food, but are also required to eat their meals at particular times. Delay of a meal will result in the patient suffering hypoglycemia.

A more recent development in insulin regimen is the basal/bolus regimen, which provides far more flexibility in quantity and timing of meals. The basal/bolus program attempts to emulate the method by which an intact pancreas controls blood glucose. Normally, the intact pancreas produces a steady supply of basal insulin to accommodate the body's basic insulin needs for glucose secreted at a relatively constant rate from the liver. The pancreas handles meals by releasing a sharp impulse of bolus insulin to accommodate a rapidly rising blood glucose resulting from transformation of carbohydrates (and, to a lesser extent, other food items, especially protein) into blood glucose.

In the basal/bolus regimen, the basal insulin releases are emulated by a once a day injection of a long acting insulin, such as Lantus®, a product of Aventis Pharmaceuticals, or Ultralente®, a product of Eli Lilly and Company. Ultralente is sometimes injected twice daily. These long acting insulins provide the body with a relatively constant supply of insulin. The bolus insulin releases are emulated by bolus injections of fast acting Humalog® (lispro), or other fast acting insulin. The amount of fast acting insulin taken in an injection must be proportional to the amount of carbohydrate taken with the meal. Some diabetics are able to further fine-tune the injection by calculating the amount of protein, which has a smaller effect on the rise of blood glucose concentration.

To illustrate the basal/bolus regimen in an example, assume a typical diabetic who requires 0.5 units per hour of basal insulin. This person will need a 12-unit injection of long acting insulin daily to cover his or her basal requirements. Timing of such an injection is not critical, and in fact, the long acting insulin is often mixed with the fast acting insulin in one of the bolus injections. Further assume that this typical diabetic's blood glucose is raised 4 mg/dl (blood glucose concentrations are measured in milligrams per deciliter) for every gram of carbohydrate eaten. This is known as carbohydrate sensitivity. Assume also that a unit of insulin (insulin is measured in “units”) reduces this typical diabetic's blood glucose concentration by 40 mg/dl. This is known as insulin sensitivity. The diabetic sits down at a meal and adds up the total grams of carbohydrates in the meal. Assume the meal consists of 80 g of carbohydrates. The diabetic would compute the increase in blood glucose concentration to be (4 mg/dl/g)*(80 g)=320 mg/dl. The diabetic would then compute the amount of bolus insulin required to accommodate, or “cover” this increase, knowing his or her insulin sensitivity. (320 mg/dl)/(40 mg/dl/unit)=8 units. The diabetic would therefore inject 8 units of fast acting insulin before eating the meal.

In practice, exercise, stress, and even unknown factors cause the above calculations to be only approximations. The diabetic, in his or her basal/bolus regimen, usually also needs to adjust the bolus dose taken based upon a blood glucose reading taken prior to the meal. A typical desired target for a diabetic's blood glucose concentration prior to a meal is 100 mg/dl. “Normal” blood glucose concentration range is 80 mg/dl to 120 mg/dl. A blood glucose concentration of 70 mg/dl or lower is usually considered to be hypoglycemic. A blood glucose concentration of 40 mg/dl is dangerously hypoglycemic and the diabetic is usually seriously impaired when his or her blood glucose concentration is at that level. A sustained blood glucose concentration of 20 mg/dl or lower is considered to expose the diabetic to permanent brain damage.

Suppose that, in the example above, the diabetic's pre-meal blood glucose concentration were 180 mg/dl. The diabetic would recognize that as being 80 mg/dl above the desired concentration of 100 mg/dl. Using the insulin sensitivity in the example, the diabetic would compute the additional insulin required as (80 mg/dl)/(40 mg/dl/unit)=2 units. In the example, the diabetic would then take a 10-unit bolus; 8 for the carbohydrates in the meal, and 2 more to “cover” the fact that the premeal blood glucose concentration was 80 mg/dl above target. If, in the example, the premeal blood glucose concentration were 80 mg/dl, the diabetic would compute a 0.5 unit negative adjustment, and thus take a bolus of 7.5 units with the meal instead of 8 units.

Insulin pumps are mechanisms that allow the basal/bolus regimen to be practiced even more effectively. An insulin pump contains a reservoir of fast acting insulin. Insulin is pumped through a tube from the reservoir into the diabetic. A computer within the pump, with which the diabetic interacts, controls the insulin pump. The diabetic programs in a “basal profile” which tells the pump how much of the fast acting insulin per unit time period to infuse into the diabetic. The pump then infuses this amount into the diabetic in a series of small infusions. In the example above, an infusion rate of 0.5 units per hour was assumed. In practice, this number varies considerably from one individual to the next. In some individuals, the rate also needs to vary during the course of a day. In particular, many diabetics find they need a higher rate of infusion for several hours before breakfast. The series of small infusions of fast acting insulin replaces the single injection of long acting insulin as described above. At a meal, the diabetic makes the same calculations described above, and interacts with the pump to cause it to infuse the proper bolus of fast acting insulin to cover the carbohydrates of the meal.

Methods and apparatus exist to assist the diabetic in the computations described. U.S. Pat. No. 5,822,715, “Diabetes management system and method for controlling blood glucose”, by Worthington, et al, (hereinafter, Worthington), is an example of the art in this field. Worthington describes a system with which the diabetic interacts, entering insulin doses, meal carbohydrate quantities, and measured blood glucose at any particular time. The system uses a measured current blood glucose concentration, insulin absorption characteristics, insulin sensitivity parameter programmed by the user, and a carbohydrate sensitivity parameter entered by the user to compute, at the time of the measurement, whether the diabetic's blood glucose concentration is above or below where it should be. Worthington's system then recommends injection or infusion of additional insulin if the blood glucose concentration is too high. If the concentration is too low, Worthington's system recommends how much additional carbohydrate should be eaten. The system warns the diabetic if the blood glucose concentration is too high or too low.

Several “continuous metering” products are currently available. One is the Glucowatch®, by the Cygnus corporation, which takes several measurements of a diabetic's blood glucose (inferred from readings of “interstitial fluid”) per hour. The Glucowatch® has the capability of warning the diabetic when his or her blood glucose concentration is too high or too low. A second such product is the Minimed Continuous Glucose Monitoring System® (CGMS), by the Medtronic Corporation, which takes even more frequent measurements than the Glucowatch®. Currently the CGMS does not alert the diabetic to high or low measurements.

A phenomena found in many diabetics is hypoglycemic rebound. If a diabetic becomes hypoglycemic, stress hormones will trigger a significant release of glucose that had been stored in the liver. This will cause a hyperglycemic event several hours after the hypoglycemic event. That is, the blood glucose concentration will swing from a low value to a high value, neither of which is healthy for the diabetic. Not every diabetic is subject to hypoglycemic rebounds, but many are. Timing of the hypoglycemic rebound also varies between individuals. One diabetic may find himself or herself to experience a rebound after two hours, while a second diabetic may not have a rebound until four hours after a hypoglycemic event. Furthermore, upon discovering the hypoglycemic event, the diabetic needs to consume some form of carbohydrate to treat the event. The system of Worthington would be valuable for telling the diabetic how much carbohydrate to consume. The diabetic could also do the calculations described above, although many times diabetics' ability to do calculations when their blood glucose concentrations are low is severely impaired. The diabetic might not be able to enter numbers into Worthington's system in his or her impaired state. The diabetic often takes more than the calculations (or, Worthington) would call for in order to more quickly get their blood glucose concentration out of the hypoglycemic range of values. Such “overtreatment” is another common cause of becoming hyperglycemic some hours after suffering a hypoglycemic event.

The diabetic frequently forgets to test his or her blood glucose concentration several hours after a hypoglycemic event, and, therefore, often only discovers a very high blood glucose concentration at a pre-meal test, which is often many hours after the hypoglycemic event occurred. Worthington and other prior art do not remind the diabetic to test their blood glucose concentration, following a hypoglycemic event, to check for a rebound or overtreatment hyperglycemic event. The Glucowatch® would provide warnings for both the original hypoglycemic event, as well as a rebound hyperglycemic event. However, the Glucowatch® is large, is expensive to purchase, and requires expensive disposables. The CGMS is also expensive, and requires a sensor to be embedded in the skin. Currently the CGMS does not give warnings for highs and lows, but could easily be modified to do so.

Therefore, there exists a need for an inexpensive blood glucose meter that reminds the user to test again, after a time interval previously programmed by the user, upon detection of a hypoglycemic event.

### SUMMARY OF THE INVENTION

A principle object of the present invention is to provide an improved, inexpensive, blood glucose meter that provides a warning to the user after a predetermined time interval following the meter's measurement of a blood glucose concentration lower than a predetermined value.

In an embodiment of the invention, the improved blood glucose meter comprises a memory programmable by the user that stores a time interval, and a blood glucose concentration limit below which a measured blood glucose concentration reading causes the time interval to be loaded into an interval timer and to start the interval timer. When the interval timer indicates that a time period equal to the time interval has elapsed, the meter alerts the user.

In an embodiment of the invention, the improved blood glucose meter will provide an audible alarm after a time interval, as described above, has expired.

In an embodiment of the invention, the improved blood glucose meter will provide a tactile alarm after a time interval, as described above, as expired.

In an embodiment of the invention, a method is described wherein an improved blood glucose meter will start a timer upon measuring a blood glucose concentration lower than a preprogrammed limit, and will produce an audible or tactile alert upon completion of a programmable time interval, to remind the user to recheck his or her blood glucose concentration.

In an embodiment of the invention, a program product is described wherein the program product when executed in a blood glucose meter, will remind the user to re-check his or her blood glucose concentration after a predetermined interval has elapsed following the blood glucose meter's measurement of a blood glucose concentration less than a predetermined threshold or limit.

### BRIEF DESCRIPTION OF THE DRAWINGS

FIG. 1 shows a drawing of a blood glucose meter and a blood glucose test strip.

FIG. 2 shows a block diagram of a blood glucose meter that implements the current invention.

FIG. 3 shows a flowchart of a program executed by the processor in the blood glucose meter that implements the current invention.

FIG. 4 shows a flowchart of a portion of a program executed by the processor in the blood glucose meter that implements the current invention. The portion shown prompts the user for information, which is then entered, by the user.

### DETAILED DESCRIPTION OF THE PREFERRED EMBODIMENTS

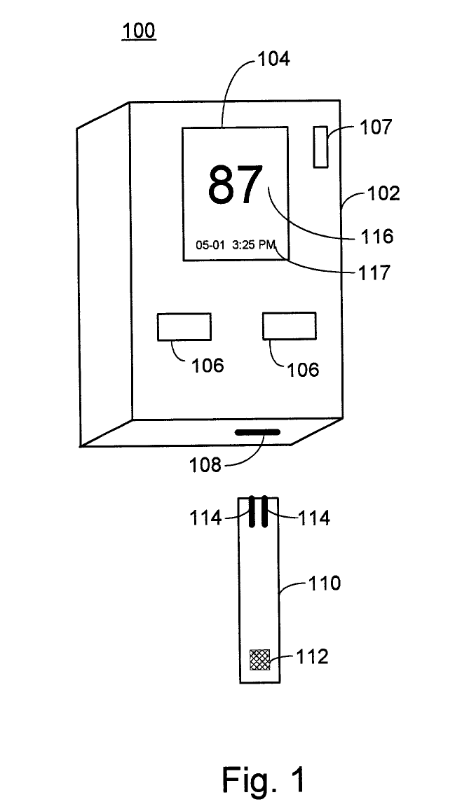

Having reference now to the figures, and in particular FIG. 1, a blood glucose meter 100 (hereinafter “meter 100”) typically has a case 102 to enclose and protect the internal components. Case 102 is typically made of plastic, but can be any suitable material. A display 104 gives the user information such as prompts for data entry, time and date, a prompt inviting the user to begin a blood glucose concentration test, as well as displaying the measured blood glucose concentration. In the exemplary FIG. 1, current reading 116 displays upon display 104, and shows an exemplary value of 87. Current time and date 117 is also displayed upon display 104.

A set of buttons 106 allows the user to input data to meter 100, turn meter 100 on or off, or to make inquiries as to previous blood glucose concentration measurements. Meters existing on the market today have widely different button 106 arrangements. Some have two buttons 106, as shown in FIG. 1. Some, such as described by Worthington, have a relatively large number of buttons 106. The particular button layout is not important to the current invention, and any pushbutton interface is intended in the scope and spirit of this invention.

Alarm 107 can be an audible alarm, a tactile alarm that vibrates, or even a blinking light.

Meter 100 has a slot 108 that receives a blood glucose test strip 110. Test strip 110 is typically a disposable item that is used for a single blood glucose concentration test and is then discarded. Typically, test strip 110 comprises a reagent area 112 upon which a sample of blood is deposited. Electrical resistance of the reagent in area 112 changes depending upon glucose concentration in the blood sample. Electrodes 114 are exposed at one end of test strip 110 in order to make electrical contact with mating electrodes (not shown) within slot 108. Each electrode 114 is electrically continuous from the exposed portion to area 112 and is electrically coupled to area 112 such that changes in resistance of the reagent can be measured at electrodes 114. Test strip 110 is inserted into slot 108 and meter 100 performs resistance measurements as a drop of blood is deposited on area 112. Meter 100 is designed to determine the blood glucose concentration of the blood sample and display the blood glucose concentration on display 104 in suitable units such as milligrams per deciliter. Other units are used in some countries, and this invention is not dependent upon the particular units used. Meter 100 could alternatively use a reagent that changes color, rather than a reagent that changes resistance. This invention is not dependent on the specific mechanism to determine the blood glucose concentration. The examples discussed are illustrative rather than limiting.

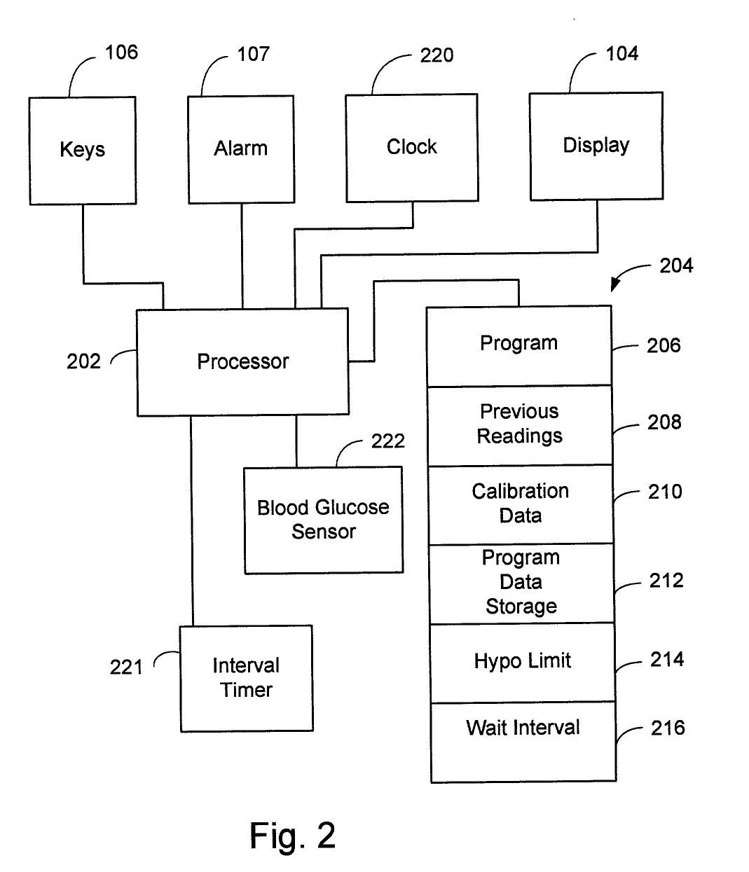

FIG. 2 shows a block diagram of a blood glucose showing the meter's functional components. Processor 202 can be any general or special purpose digital processor that can be suitably programmed to perform the input/output (I/O) needs of the meter, as well as all required computations and control.

Electrically coupled to processor 202 is a memory 204. Memory 204 can be any suitable memory such as Static Random Access Memory (SRAM), Dynamic Random Access Memory (DRAM), flash memory, ferroelectric memory, or magnetic memory. Not all portions of memory 204 need be of the same type.

Program storage 206 holds the executable program used by processor 202 to perform the control and computational steps required for the function of the meter. Program storage 206 can be made of Read Only Memory (ROM), since the program may not need to be changed once written and debugged. Advantageously, however, program storage 206 is implemented in a nonvolatile memory capable of both reading and writing, such as Flash memory or Ferroelectric memory. Such memory allows more flexibility during manufacturing, and would allow for modifying the program after manufacture.

Most meters on the market store a history of some number of the most recent previous tests, comprising a number of prior blood glucose concentration results, which are also stored in a portion of memory 204 called previous readings 208. For example, a meter might have enough storage in previous readings 208 to store a history of the last 100 tests of blood glucose concentrations, along with the month, day, and time at which those tests were performed. Some meters have the capability of downloading this history to a computer at the user's home or at a doctor's office. Computer analysis of the history can then be performed by the user or the doctor to look for trends or trouble spots in the therapy. Previous readings 208 can be advantageously implemented in nonvolatile memory. Previous readings 208 could also be implemented in volatile memory such as SRAM or DRAM, since only the test history would be lost if the battery (not shown) which powers the meter should fail.

Calibration data 210 is stored in meter's memory 204. The reagent on the test strips can vary slightly from lot to lot during manufacture of the test strips. Most meters have “fine tuning” data shipped with each group of test strips. Some meters provide a semiconductor chip product containing this data in nonvolatile form. This chip effectively becomes calibration data 210 as a memory portion of the aggregate memory 204. Some meters provide a test strip that contains the calibration data that is inserted into slot 108 of FIG. 1, read by processor 202, and stored in nonvolatile memory storage as calibration data 210 by the processor 202.

Memory 204 further contains program data storage 212, which is used for temporary storage of numbers needed during calculations and processing by processor 202 as it executes the steps programmed in program storage 206. Data storage 212 can be implemented in either volatile storage such as SRAM or DRAM or in nonvolatile storage, as described above.

Memory 204 further contains hypo limit 214, a number entered by the user to define his or her hypoglycemic limit. The user knows from experience what low blood glucose concentration limit, or threshold, will generally produce a hypoglycemic rebound. As described in more detail below, if a measurement of blood glucose concentration is below the value in hypo limit 214, the meter will produce an alarm at a programmable time interval thereafter. Hypo limit 214 is preferably implemented with nonvolatile storage so that the user does not have to reenter the value stored in hypo limit 214 if the battery (not shown) powering the meter should fail.

Memory 204 further contains storage for wait interval 216 that holds a value of time that the meter will wait after measuring a hypoglycemic event, after which an alarm will be actuated, as described in detail below. The user knows from experience when a hypoglycemic rebound will occur following a hypoglycemic event. The user will enter a value into wait interval 216 that is appropriate for the user. Typically, a time of two to six hours would be entered and stored in wait interval 216.

Blood glucose sensor 222 is a device that measures blood glucose concentration of a sample of blood. As described above, sensor 222 could measure blood by resistivity measurements of reagent 112, or color change of reagent 112, or any other method of determining the blood glucose concentration of a sample of blood. Sensor 222 is electrically coupled to processor 202 so that the blood glucose concentration measurement can be transmitted to processor 202.

Keys 106 are electrically coupled to processor 202. Keys 106 are used to turn the meter 100 on or off, and allow the user to enter data or commands to processor 202. For example, the values stored in hypo limit 214 and wait interval 216 would be entered on keys 106, in a conventional manner.

Alarm 107 is electrically coupled to processor 202. Alarm 107 could be an audible alarm. Alarm 107 could be a tactile alarm that vibrates or shakes when activated by processor 202. Alarm 107 could be a visible alarm, implemented with a light emitting diode (LED), an incandescent light, or any other visible means of alerting the user.

Display 104 is electrically coupled to processor 202, and communicates information to the user. Information such as date, time, blood glucose concentration, and prompts for data entry are advantageously displayed on display 104. Display 104 is typically implemented as a liquid crystal display (LCD) but could be an array of LEDs.

Clock 220 is electrically coupled to processor 202. Clock 220 is a conventional clock that provides hour/minute, day, and month capabilities. This information is needed to document the time when blood glucose measurements are taken.

Interval timer 221 is a timing device used to indicate the elapse of time periods. “Egg timers”, and common kitchen timers are examples of interval timers familiar to most people. Interval timer 221 can be initialized to a value. Upon receiving a signal to start, interval timer 221 begins counting. The count may increment or decrement. In one embodiment, interval timer 221 is initialized with the value stored in wait interval 216. Interval timer 221 is then started in a decrementing mode. Upon the interval timer function reaching a predetermined value, advantageously zero, processor 202 is signaled over the electrical coupling between interval timer 221 and processor 202. Processor 202 then activates alarm 107 for some predetermined time, or until the user shuts off alarm 107 using keys 106. As will be appreciated by those skilled in the art, many variants of this mechanism are possible. For example, in a second embodiment, interval timer 221 could be loaded with the value stored in wait interval 216, be initialized to a predetermined value, advantageously zero, and then be incremented until the counter value equals or exceeds the value loaded from wait interval 216. In another embodiment, interval timer 221 could be initialized to zero, or other predetermined value, and started counting by processor 202. Processor 202 periodically would then periodically receive a value from interval timer 221 indicating how much time has elapsed on the timer since it was started. Processor 202 would compare that value with the value stored in wait interval 216. Processor 202 would then activate alarm 107 when the value from the timer exceeds the value stored in wait interval 216.

Interval timer 221 could be implemented as a feature of clock 220. Many digital clocks also have interval timer functions. Interval timer 221 is shown in the figure as a separate block for clarity.

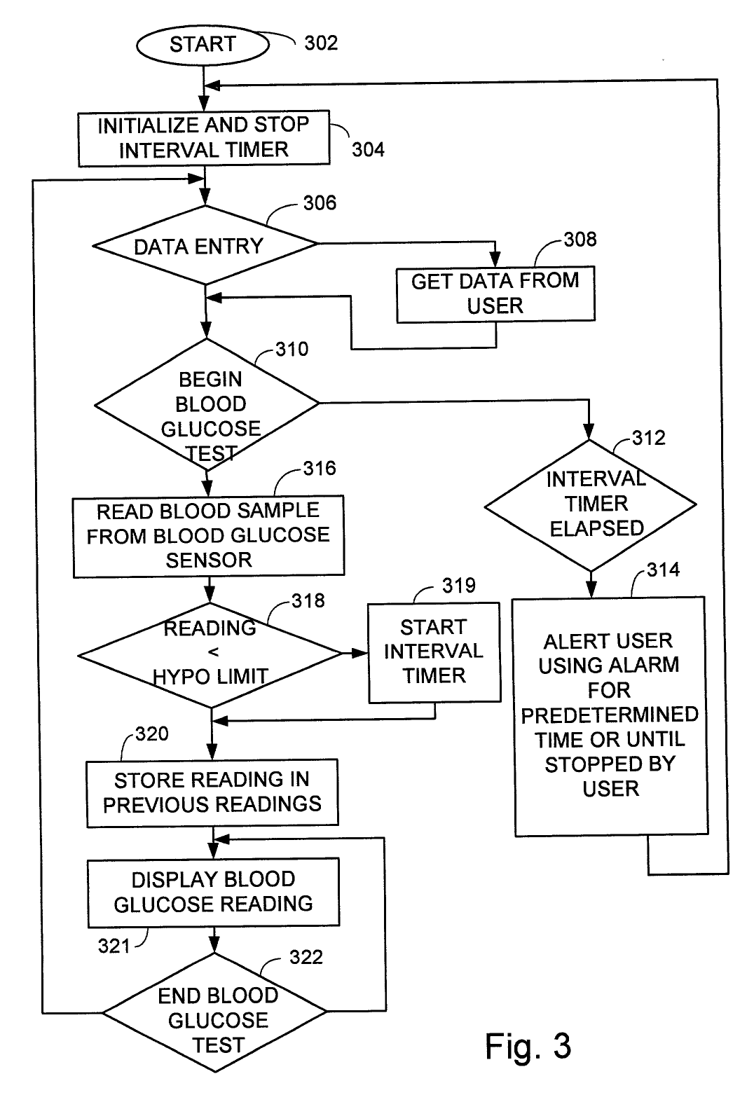

FIG. 3 shows an exemplary flowchart of the steps that are executed by processor 202 under control of the program stored in program 206.

Block 302 is the beginning of the process and simply passes control to block 304. Block 304 initializes interval timer 221 to a predetermined value and makes sure the interval timer 220 is not running. Block 304 passes control to block 306.

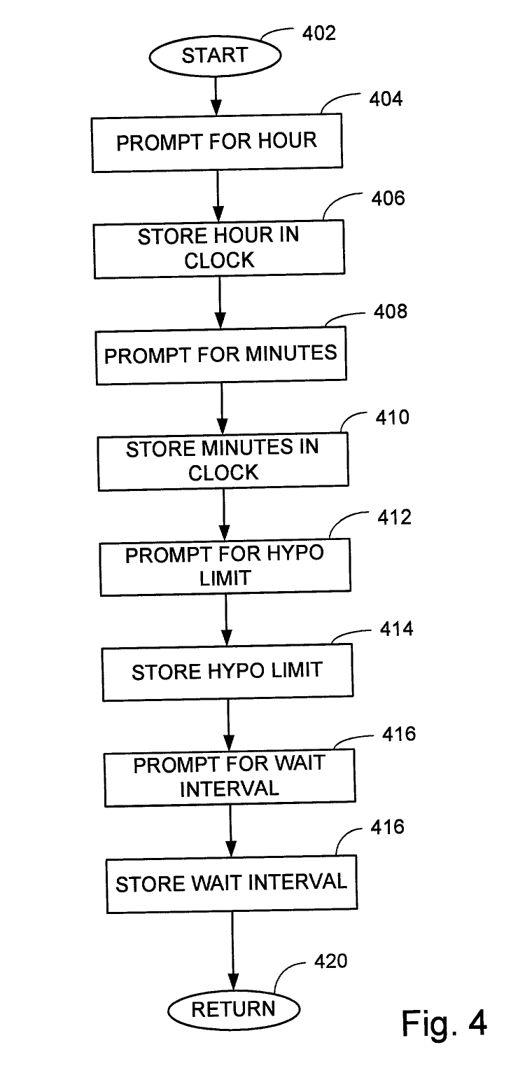

Block 306 checks to see if the user wants to enter data. Some meters have a special key 106 for this purpose. Some meters with only two keys 106 indicate that the user wants to enter data by pressing both keys 106 simultaneously. If the user does want to enter data, control is passed to block 308, which receives the user's data entry. An exemplary set of steps executed by block 308 is shown in FIG. 4. After data has been received from the user in block 308, or, if no data entry was desired in block 306, control is passed to block 310.

Block 310 checks to see if a measurement, or test, of a blood sample is desired. Most meters begin a test when a strip 110 is inserted into slot 108, although other meters can and do use other means to signal a beginning of a test. If a test is not desired, control is passed to block 312; otherwise, control is passed to block 316.

Block 316 is the step in which blood glucose sensor 222 determines the blood glucose concentration and communicates that value over the electrical coupling to processor 202. Control then passes to block 318, where processor 202 compares the value of the blood glucose concentration with the value stored in hypo limit 214. If the value of the blood glucose concentration is less than the value stored in hypo limit 214, control passes to block 319; otherwise control is passed to block 320.

In block 319, processor 202 fetches the value from wait interval 216, stores the value in interval timer 221, and activates interval timer 221. In this example, interval timer 221 decrements. As described earlier, interval timer 221 could also count up from zero to the value in wait interval 216, as a variant of the implementation described. The scope of this invention includes any timer mechanism for interval timer 221. The particular details of loading and sensing interval timer 221 will vary depending on the exact mechanism employed. Block 319 passes control to block 320 upon completion.

In block 320, processor 202 stores the measured blood glucose concentration, together with the time and date of the measurement, in previous readings 208. Control then passes to block 321.

In block 321, processor 202 displays the measured blood glucose concentration. Other information such as date and time can also be displayed on display 104. Control passes then to block 322.

Block 322 continues to display the blood glucose concentration on display 104 until the end of the blood glucose test is signaled. The signal could be the withdrawal of test strip 110 from slot 108. The signal could be driven by a separate counter (not shown) that limits the duration of the test to save battery power. Power saving timeouts are well known in currently available blood glucose meters. Upon end of the blood glucose concentration test, control is passed from block 322 to block 306.

Block 312 checks if the time interval initialized in interval timer 221 has elapsed. In the example, block 319 initialized interval timer 221 with the value stored in wait interval 216 and started the timer decrementing. Expiration of the time period specified by the value of the wait interval 216 can be indicated by processor 202 comparing the value of interval timer against a predetermined value, advantageously zero. Some embodiments of interval timer 221 could activate an interrupt signal coupled to processor 202. If the interval has elapsed, control is passed to block 314; otherwise, control is passed to block 306.

In block 314, processor 202 activates alarm 107 for a predetermined time period, or until the user deactivates alarm 107 by using one or more keys 106, according to the particular implementation's choice of a deactivation keystroke or keystroke sequence. Block 314 then transfers control to block 304.

FIG. 4 shows an exemplary set of steps by which the user can enter data into the meter.

Block 402 is the starting block, to which control is passed from block 306 of FIG. 3. Block 402 passes control to block 404.

Block 404 prompts the user to enter the present time-of-day hour. This is usually done by displaying “0” on display 104 and incrementing the hour for each push of a key 106. When the correct hour is reached, the user pushes a different key 106 to verify that the correct hour is displayed. Upon the user's verification, block 406 stores the hour in storage (not shown) in clock 220.

Similarly to blocks 404 and 406 for entering and storing the correct hour, blocks 408 and 410 prompt for, and store, the correct minutes of the current time.

Additional similar steps (not shown) are usually added to prompt for, and store, month and date information into storage (not shown) in clock 220.

Block 412 prompts the user for a value for hypo limit 214. A zero value would be displayed. This value would be incremented, as described above, each time a key 106 is pushed. When the desired value for hypo limit 214 is displayed, the user would push a different key 106 to verify that the correct hypo limit is displayed. Upon the user's verification, block 414 stores the value into hypo limit 214.

Block 416 prompts the user for a value for wait interval 216. A zero hour value would be displayed. This value would be incremented, as described above, each time a key 106 is pushed. When the desired wait time is displayed, the user would push a different key 106 to verify that the correct wait time is displayed. Upon the user's verification, block 416 stores the value into wait interval 416.

The routines, or sequences of instructions, executed on processor 202 to implement the embodiments of the invention, are stored in program 206 in memory 204. These routines are simply referred to as “computer programs”, or simply “programs”. The computer programs typically comprise one or more instructions that are resident in program storage 206, and that, when read and executed by processor 202, cause processor 202 to perform the steps necessary to execute steps or elements embodying the various aspects of the invention. Moreover, while the invention has been described in the context of a fully functioning blood glucose meter, those skilled in the art will appreciate that the various embodiments of the invention are capable of being distributed as a program product in a variety of forms that can be written into program storage 206, and that the invention applies equally regardless of the particular type of signal bearing media used to actually carry out the distribution. Examples of signal bearing media include but are not limited to recordable type media such as volatile and non-volatile memory devices, floppy and 15 other removable disks, hard drives, magnet tape, optical disks, among others, and transmission type media such as digital and analog communication links.

While the present invention has been described with reference to the details of the embodiments of the invention shown in the drawings, these details are not intended to limit the scope of the invention as claimed in the appended claims.

What is claimed is:

1. A blood glucose meter that provides a warning to the user after a predetermined time interval following the meter's measurement of a blood glucose concentration lower than a predetermined value.

2. A blood glucose meter comprising:
a digital processor;
a memory, coupled to the processor, capable of storing an executable program, program data storage, and data entered by a user;
a blood glucose sensor, coupled to the processor, capable of analyzing a first sample of blood and reporting to the processor a blood glucose concentration of the first blood sample;
a plurality of keys coupled to the processor, by which the user can enter data;
an alarm, coupled to the processor;
a display, coupled to the processor; and
an interval timer;
wherein the blood glucose meter is capable of reminding the user to re-test the user's blood glucose concentration, using a second blood sample, after a pre-programmed time period following a hypoglycemic measurement of the first blood sample.

3. The blood glucose meter of claim 2, wherein the memory stores a user-entered value for a hypo limit, the hypo limit being a threshold value of the blood glucose concentration below which the user has determined a rebound hyperglycemic event will occur as a result; and a value for a wait interval, the wait interval being a time period the user has determined is appropriate to wait, after a blood glucose concentration measurement, of the first blood sample, below the hypo limit, before being reminded to re-test, using the second blood sample, for a rebound hyperglycemic measurement.

4. The blood glucose meter of claim 2, wherein the memory comprises nonvolatile storage.

5. The blood glucose meter of claim 2, wherein the blood glucose sensor makes a plurality of resistive measurements of a reagent area to determine the blood glucose concentration.

6. The blood glucose meter of claim 2, wherein the blood glucose sensor makes a plurality of color measurements of a reagent area to determine the blood glucose concentration.

7. The blood glucose meter of claim 2, wherein the alarm produces an audible sound.

8. The blood glucose meter of claim 2, wherein the alarm produces a tactile warning capable of being felt by the user.

9. The blood glucose meter of claim 2, wherein the alarm produces a visual warning to the user.

10. The blood glucose meter of claim 2, wherein the display is a liquid crystal display, or a light-emitting diode display.

11. A method of reminding a diabetic to re-check his or her blood glucose concentration after a predetermined wait period following a hypoglycemic event, implemented in a blood glucose meter, comprising the steps of:
receiving a hypo limit value from the user and saving the limit in a memory;
receiving a wait interval from the user and saving the interval in a memory;
measuring a blood sample and determining a blood glucose concentration of the blood sample;
comparing the blood glucose concentration of the blood sample to the hypo limit value; and
if the blood glucose concentration of the blood sample is less than the hypo limit value, reminding the diabetic to re-check his or her blood glucose concentration after waiting a time period equal to the value of the wait interval.

12. The method of claim 10, wherein the step of reminding the diabetic to re-check his or her blood glucose concentration comprises activating an audible alarm.

13. The method of claim 10, wherein the step of reminding the diabetic to re-check his or her blood glucose concentration comprises activating a tactile alarm.

14. The method of claim 10, wherein the step of reminding the diabetic to re-check his or her blood glucose concentration comprises activating a visual alarm.

15. A program product, comprising:
a program configured to run in a blood glucose meter, the program comprising steps that will compare a measurement of blood glucose concentration to a value stored in a hypo limit, and if the measurement is less than the value, will cause an alarm to be activated after a predetermined interval following the measurement; and
a signal bearing medium bearing the program.
